(19) United States
(12) Patent Application Publication
(10) Pub. No.: US 2022/0280645 A1
(43) Pub. Date: Sep. 8, 2022

(54) Pharmaceutical Eutectic Salt Formation

(71) Applicant: SeraNovo Holding B.V., Leiden (NL)

(72) Inventors: Niall Hodgins, Leiden (NL); Bastiaan Kluft, Leiden (NL); Christian Degeling, The Hague (NL)

(21) Appl. No.: 17/636,390

(22) PCT Filed: Aug. 18, 2020
(86) PCT No.: PCT/NL2020/050515

§ 371 Date: Feb. 18, 2022

(30) Foreign Application Priority Data
Aug. 19, 2019 (NL) 2023661

(51) Int. Cl. A61K 47/12; A61K 9/00; A61K 47/10; A61K 47/18; A61K 47/26; A61K 47/34; A61K 47/38

(52) U.S. Cl. CPC A61K 47/12; A61K 9/0053; A61K 47/10; A61K 47/186; A61K 47/26; A61K 47/34; A61K 47/38

(57) ABSTRACT

The invention is directed to a pharmaceutical composition that is liquid at 37° C. and 1 atm, comprising a eutectic mixture of at least an active pharmaceutical ingredient (API) salt, a hydrophilic pharmaceutically acceptable eutectic constituent and a polymer solubilizer, and further comprising a precipitation inhibitor (PI). In another aspect, the invention is directed to a method for preparing such a composition.

The invention is in the field of pharmaceutical formulations. In particular, the invention is directed to a process to prepare a pharmaceutical formulation based on eutectic mixtures and formulations obtainable with this process.

Over the last decade, there has been an increased interest in formulation technologies to increase drug solubility and bioavailability. About 40% of the drugs in the discovery pipeline are failing out due to poor solubility with massive inherent costs and lengthy time delays in getting to market. Thus, there is a clear need to develop methods to make drugs more soluble. Several approaches have been attempted to solve this problem such as formulating the drug with polymers, solubilizing the drug in lipids prior to administration, micronizing the drug particles and crystal engineering (e.g. salts).

The oral route is the most preferable route of active ingredient administration because of several benefits, such as better patient compliance, safety and versatility. Therefore, a vast number of active ingredients are formulated in oral pharmaceutical dosage forms and mostly as solid pharmaceutical preparations, e.g. tablets, chewable tablets, capsules. However, there are some obstacles and difficulties when developing solid forms for oral administration, as many active pharmaceutical ingredients exhibit a low solubility in aqueous fluids which leads to decreased dissolution rate and limited therapeutic effect. The aqueous solubility of an active ingredient is one of the most important physicochemical properties as low aqueous solubility and low dissolution rate can reduce the active ingredient absorption in the gastro-intestinal tract. Low active ingredient solubility also directs to decreased bioavailability, increased chance of food effect, more frequent incomplete release from the dosage form and higher interpatient variability.

Poorly water soluble active pharmaceutical ingredients, typically compounds having solubility in water below 0.1 mg/ml, count for the large majority of the pharmaceutical active ingredients, thereby limiting their potential uses and increasing the difficulty of formulating bioavailable pharmaceutical products. In this category, example active ingredients include Itraconazole, Cyclosporine A, Carvedilol and Griseofulvin. Poorly soluble active ingredients have stimulated the development of active ingredient delivery technologies to overcome the obstacles to their solubilization through either chemical or mechanical modification of the environment surrounding the active ingredient molecule, or physically altering the macromolecular characteristics of aggregated active ingredient particles. These technologies include both traditional methods of solubility enhancement, such as particle size reduction, addition of surfactants and inclusion in cyclodextrin-active ingredient complexes, and the use of more novel mechanisms such as self-emulsifying systems, micronisation via nanoparticles, pH adjustment and salting-in processes.

Various methods are already known for the industrial preparation of dosage forms for oral administration comprising active pharmaceutical ingredients having low solubility. However, in the art substantial difficulties in the production of oral solid formulations of a desirable bioavailability have been encountered because of the very poor solubility of said active ingredient.

Poorly water-soluble APIs require a dedicated formulation technology in order to be effectively absorbed in the gastrointestinal (GI) tract when orally administered. Without the APIs dissolving in aqueous solutions at systemic pH ranges, the absorption of APIs will be very variable and poor which affects the safety and limits the therapeutic effects of the APIs.

A particular formulation technology that has been suggested in the art involves the formulation or dissolution of the API in a deep eutectic solvent (DES). This approach however, is still limited in the type of constituents that can be used to make the DES to solubilize the API at a sufficiently high concentration. For example, a basic API may require an acidic DES to achieve a high concentration of 200 mg/ml, but the acid in the acidic DES may degrade the API over time making it unsuitable for actual use.

Other approaches, that are similarly based on the principle of eutectic systems, involve the formation of a eutectic mixture that is based on at least the API itself (see e.g. US2007224261 and Aroso et al. International Journal of Pharmaceutics 492 (2015) 73-79). However, these approaches still suffers from an undesirably low bioavailability, i.a. because upon mixing of the eutectic mixture with an aqueous environment, e.g. gastrointestinal fluid, the active pharmaceutical ingredient separates from the mixture, thereby limiting absorption by the patient body.

It is an object of the present invention to improve the solubility and bioavailability characteristics of poorly water soluble active ingredients.

The present inventors have found that this object can be met by providing a eutectic mixture that is based on at least a salt form of an API, in combination with a precipitation inhibitor (PI). It was found that the specific combination of the salt and the precipitation inhibitor result in both a good solubility and bioavailability.

Moreover, advantageously, the inventors found that by using a hydrophilic pharmaceutically acceptable eutectic constituent (herein further also referred to as simply eutectic constituent) as a second constituent to form the eutectic mixture (the first constituent being the API salt), the composition typically immediately releases the API after administration, in contrast to a slow release. This is also believed to attribute to the favorable bioavailability properties of the present composition.

Further, the inventors found that the bioavailability can be improved by providing the pharmaceutical composition as a liquid (at physiological conditions, i.e. 37° C. and 1 atm), which is preferred.

Accordingly, the present invention is directed to a pharmaceutical composition that is preferably liquid at 37° C. and 1 atm and which is based on a eutectic mixture of at least an active pharmaceutical ingredient (API) salt, a hydrophilic pharmaceutically acceptable eutectic constituent and a polymer solubilizer, and which composition further comprises a precipitation inhibitor (PI).

In another aspect, the invention is directed to a method to prepare the pharmaceutical composition that is preferably liquid at 37° C. and 1 atm, said method comprising a eutectic mixture of at least an active pharmaceutical ingredient (API) salt, a hydrophilic pharmaceutically acceptable eutectic constituent and a polymer solubilizer; and mixing said eutectic mixture with a precipitation inhibitor (PI) to form said pharmaceutical composition.

Advantageously, because the eutectic mixture is based on the API itself, very high concentrations of the API is achievable, for instance even up to 580 mg/ml. In typical compositions however, such a high concentration is not preferable due to concomitant hampering of the bioavailability. As such, the API is preferably present in the composition in a concentration of at least 50 mg/ml, preferably at least 100 mg/ml, more preferably at least 150 mg/ml. However, lower concentrations than 50 mg/ml, for instance in the range of 1 to 25 mg/ml are also well possible.

As meant herein, a eutectic mixture is a homogeneous mixture of constituents having a lower melting point than the melting points of each of the constituents of this mixture. The composition of the present invention preferably has a melting point at 1 atm of 37° C. or lower, i.e. it is liquid at physiological conditions. Thus the composition may be solid at 20° C. and 1 atm, and liquefies only after administration to the body. In a preferred the embodiment, the eutectic mixture itself is also a liquid at physiological conditions and has a melting point at 1 atm of 37° C. or lower.

The liquid state of the composition according to the present invention can be distinguished from known composition having a gel state, as for example disclosed in WO 2011/014850. The present composition is liquid and thus fluidic, unlike gels that exhibits no flow when in the steady-state. In fact, the role of the polymer solubilizer according to the present invention is to prevent gel-formation.

One of the constituents on which the eutectic mixture according to the present invention is based is the API salt. Salt formation of APIs is generally well-known in the art, also to increase solubility and dissolution rates. The counter ion of the salt can change the pH at the dissolving surface of a salt particle in the diffusion layer, resulting in a higher dissolution rate of the salts compared with that of the corresponding free forms. According to the Henderson-Hasselbalch equations, the change of pH highly influences the aqueous solubility of an ionizable drug. In theory, the solubility of a weak basic drug increases exponentially with decreasing pH at the pH range between its pKa and pH max (pH of maximum solubility in the pH-solubility profile).

The API salt can be generated by reacting the API with the salt forming substance to create the salt of the active pharmaceutical ingredient. For the present invention, it is preferred that for basic APIs, e.g. a weak basic drug such as propranolol, cetirizine or diphenhydramine, the API salt according to the present invention is formed by said API and an acid having a pKa value that is lower than 3 with respect to the pKa of the conjugated acid of the basic API. Suitable acids are preferably selected from the group consisting of hydrochloric acid, sulfuric acid, phosphoric acid, maleic acid, L-tartaric acid, fumaric acid, citric acid, glycolic acid, malic acid, hippuric acid, lactic acid, succinic acid, adipic acid, sebacic acid, acetic acid, p-toluenesulfonic acid, methanesulfonic acid, benzenesulfonic acid, oxalic acid, malonic acid, gentisic acid, benzoic acid and nicotinic acid. In particularly preferred embodiment, the acid comprises hydrochloric acid.

In the embodiments of the present invention wherein the API salt is based on an acidic API, e.g. a weak acidic drug such as ibuprofen or phenytoin, and a base, it is preferred that a base is used of which the conjugated acid has a pKa value of more than 7 or above. Suitable bases are preferably selected from the group consisting of sodium hydroxide, potassium hydroxide, sodium carbonate, potassium carbonate, sodium glycinate, sodium lysinate, sodium glycinate monohydrate, N-methylglucosamine, potassium glycinate and tribasic sodium and potassium phosphates. In particularly preferred embodiment, the base comprises sodium hydroxide or potassium hydroxide.

The composition of the API salt, and in particular the structure of the counter ion, can influence the properties of the API salt and the suitability of the salt to form the eutectic mixture. The formation of a eutectic mixture is typically based on the presence of a hydrogen bonding donor and acceptor interaction associated with API salt and the eutectic constituent. As such, preferably a configuration exists for a stable complex associated with a eutectic mixture that involves a strong interaction between an electron donor group within one of the two components A or B such as a carbonyl oxygen, amidyl nitrogen, or etheral oxygen with alkyl proton, hydroxyl proton, amine proton or a sigma acceptor group such as chlorine. Without wishing to be bound by any particular theory, it is believed that a small (e.g. atomic) counter ion e.g. the Cl in HCl or in choline chloride can facilitate the strong intermolecular forces required to depress the melting point of the API salt and the eutectic constituents to create a liquid at 37° C. For this reason, preferably either the API salt has a small counter ion capable of forming strong intermolecular bonds or the eutectic constituent has such a moiety.

Accordingly, in embodiments wherein the API salt is based on a basic API, the acid which with the API salt is formed preferably comprises an atomic anion (e.g. a halide anion), while said pharmaceutically acceptable eutectic constituent comprises one or more functional groups capable of hydrogen bonding. Alternatively, in the embodiment wherein the acid which with the API salt comprises a molecular anion, and said pharmaceutically acceptable eutectic constituent comprises an atomic anion or a small (e.g. atomic) moiety capable of forming strong intermolecular bonds.

Formation of a eutectic mixture of an API salt and principles behind this concept is also described in US 2007/224261 and WO 2014/145156, which are incorporated herein in their entirety.

As used herein, the terms “drugs,” “active ingredients,” and “active pharmaceutical ingredients” are used interchangeably.

As mentioned herein-above, the invention intends to deal with poorly water soluble API. The API may for example have an aqueous solubility of not more than 1 mg/mL at pH 6.8 when it is a weakly basic compound, of no more than 1 mg/mL at pH 1.2 when it is a weakly acidic compound, and of no more than 1 mg/mL at any pH between the physiological pH of 1.0-8.0 for neutral or non-ionisable compounds. The solubility of an API can be determined by adding the highest dose strength in 250 mL of aqueous solutions with a pH ranging from 1 to 7.4 to cover GI physiological conditions. If the highest dose strength of API is not dissolved in 250 mL of solution of any pH from 1-7.4, the API is considered to be poorly water soluble.

As used herein, the term “weakly basic compound”, as well as reference to any specific new chemical entity, drug, or active pharmaceutical ingredient, includes the base, pharmaceutically acceptable salts, polymorphs, stereoisomers, solvates, esters and mixtures thereof, which is a chemical base in which protonation is incomplete in an aqueous medium. In one embodiment, the weakly basic compound of the compositions of the present invention can refer to a compound having at least one pKa in the range of less than 14, wherein pKa can be measured or by calculation. In another embodiment, the weakly basic compound of the compositions of the present invention can refer to a compound having at least one pKa of less than 14, which has a pH dependent solubility between physiological pH with a lower solubility at higher pH. In another embodiment, the weakly basic drug of the compositions of the present invention can refer to a compound having at least one pKa of 0.0-10.0, which has a pH dependent solubility between physiological pH of 1.0-8.0 with a lowest solubility at around pH 6.0-8.0. In another embodiment, the weakly basic compound has a solubility of not more than about 1 mg/mL at pH 6.8. In another embodiment, the weakly basic compound includes at least one basic nitrogen atom. In yet another embodiment, the weakly basic compound has a pKa of less than 14, and a solubility of not more than about 1 mg/mL at pH 6.8. In yet another embodiment, the weakly basic compound has a pKa of less than 14, and includes at least one basic nitrogen atom. In yet another embodiment, the weakly basic compound as a pKa of less than 14, a solubility of not more than 1 mg/mL at pH 6.8, and includes a least one basic nitrogen atom.

As used herein, the term “weakly acidic compound” as well as reference to any specific new chemical entity, drug, or active pharmaceutical ingredient, includes the acid, pharmaceutically acceptable salts, polymorphs, stereoisomers, solvates, esters and mixtures thereof, which is a chemical base in which deprotonation is incomplete in aqueous medium. In one embodiment, the weakly acidic drug of the compositions of the present invention can refer to a compound having at least one pKa of less than 14, wherein pKa can be measured or by calculation. In another embodiment, the weakly acidic compound of the compositions of the present invention can refer to a compound having at least one pKa of less than 14, which has a pH dependent solubility between physiological pH with a lower solubility at lower pH. In another embodiment, the weakly acidic drug of the compositions of the present invention can refer to a compound having at least one pKa of 0.0-10.0, which has a pH dependent solubility between physiological pH of 1.0-8.0 with a lower solubility around pH 1.0-2.0. In another embodiment, the weakly acid compound has a solubility of not more than about 1 mg/mL at pH 1.0-2.0. In another embodiment, the weakly acidic compound includes at least one acidic functional group. In yet another embodiment, the weakly acidic compound has at least one pKa of less than 14, and a solubility of not more than about 1 mg/mL at pH 1.2. In yet another embodiment, the weakly acidic compound has a pKa of less than 14, and includes at least one acidic functional group. In yet another embodiment, the weakly acidic compound has a pKa of less than 14, a solubility of not more than 1 mg/mL at pH 1.2, and includes a least one acidic functional group.

Suitable drugs for preparing the composition according to the present invention include, but are not limited to, members of the therapeutic categories analgesics, anti-inflammatory agents, anthelmintics, antiarrhythmic agents, anti-bacterial agents, anti-viral agents, anticoagulants, anti-depressants, anti-diabetic agents, anti-epileptic agents, anti-fungal agents, anti-gout agents, antihypertensive agents, anti-malarial agents, anti-migraine agents, anti-muscarinic agents, anti-neoplastic agents, erectile dysfunction improving agents, immunosuppressants, anti-protozoa agents, anti-thyroid agents, anti-anxiolytic agents, sedatives, hypnotics, neuroleptics, 6-blockers, cardiac inotropic agents, corticosteroids, diuretics, antiparkinsonian agents, gastrointestinal agents, histamine receptor antagonists, keratolytics, lipid regulating agents, anti-angina agents, cox-2 inhibitors, leucotriene inhibitors, macrolides, muscle relaxants, nutritional agents, opioid analgesics, protease inhibitors, sex hormones, stimulants, anti-osteoporosis agents, anti-obesity agents, cognition enhancers, anti-urinary incontinence agents, nutritional oils, anti-benign prostate hypertrophy agents, essential fatty acids, non-essential fatty acids, and any combinations of two or more thereof.

Specific examples of suitable active pharmaceutical ingredients include, but are not limited to: abiraterone, acutretin, albendazole, albuterol, aminogluthemide, amiodarone, amlodipine, amphetamine, amphotericin B, atorvastatin, atovaquone, azithromycin, baclofen, beclomethsone, benezepril, benzonatate, betamethasone, bicalutanide, boceprevir, budesonide, bupropion, busulphan, butenafine, calcifediol, calciprotiene, calcitriol, camptothecan, candesartan, capsaicin, carbamezepine, carotenes, celecoxib, cerivistatin, cetrizine, chlorpheniramine, cholecalciferol, cilostazol, cimetidine, cinnarizine, ciprofloxacin, cisapride, clarithromycin, clemastine, clomiphene, clomipramine, clopidrogel, codeine, coenzyme Q10, cyclobenzaprine, cyclosporine, danazol, dantrolene, dexchlophenir amine, diclofenac, dicoumarol, digoxin, dihydroepiandrosterone, dihydroergotamine, dihydrotachysterol, dirithromycin, donepezil, efavirenz, eposartan, ergocalciferol, ergotamine, essential fatty acid sources, eszopiclone, etodolac, etoposide, famotidine, fenofibrate, fentanyl, fexofenadine, finasteride, flucanazole, flurbiprofen, fluvastatin, fosphenytion, frovatriptan, furazolidone, gab apentin, gemfibrozil, glibenclamide, glipizide, glyburide, glymepride, griseofulvin, halofantrine, ibuprofen, irbesartan, irinotecan, isosorbide, isotreinoin, itraconazole, ivermectin, ketoconazole, ketorolac, lamotrigine, lanosprazole, leflunomide, lisinopril, loperamide, loratadine, lovastatin, L-thryroxine, lutein, lycopene, medroxyprogesterone, mefepristone, mefloquine, megesterol, metaxalone, methadone, methoxsalen, metronidazole, metronidazole, miconazole, midazolam, miglitol, minoxidil, mitoxantrone, montelukast, nabumetone, nalbuphine, naratiptan, nelfinavir, nifedipine, nilsolidipine, nilutanide, nitrofurantoin, nizatidine, omeprazole, oprevelkin, osteradiol, oxaprozin, paclitaxel, paricalcitol, paroxetine, pentazocine, pioglitazone, pizofetin, pravastatin, prednisolone, probucol, progesterone, pseudoephedrine, pyridostigmine, rabeprazole, raloxifene, refocoxib, repaglinide, rifabutine, rifapentine, rifaximine, rimexolone, ritanovir, rizatriptan, rosiglitazone, saquinavir, sertraline, sibutramine, sildenafil, simvastatin, sirolimus, spironolactone, sumatriptan, tacrine, tacrolimus, tamoxifen, tamsulosin, targretin, tazarotene, telaprevir, telmisartan, teniposide, terbinafine, terzosin, tetrahydrocannabinol, tiagabine, ticlidopine, tirofibr an, tizanidine, topiramate, topotecan, toremifene, tramadol, tretinoin, troglitazone, trovafloxacin, ubidecarenone, valsartan, venlafaxine, vertoporfin, vigabatrin, vitamin A, vitamin D, vitamin E, vitamin K, zafirlukast, zileuton, ziprasidone, zolmitriptan, Zolpidem, and zopiclone. This listing is not intended to be exhaustive, as many other drug substances can be used. Also, any of the pharmaceutically acceptable salts, esters, solvates, hydrates and other derivatives that can deliver any of the drugs also can be used, in any polymorphic forms, and combinations of any two or more active ingredients can be used to prepare formulations. Although many of the drugs are commonly formulated using their pharmaceutically acceptable derivatives such as salts and esters, for the sake of brevity only the base drugs have been listed.

The eutectic constituent that is further used to form the eutectic mixture with the API salt is hydrophilic. As such, the composition can rapidly dissolve in an aqueous environment. An “aqueous environment” as used herein generally means a gastrointestinal fluid if in vivo and an aqueous test medium if in vitro. More specifically, “aqueous environment” encompasses, if the aqueous environment is in vivo and has a pH in the range of 1.0 to 2.0, the stomach; and if the aqueous environment is in vivo and has a pH in the range of 5.0 to 8.0, the intestine. The eutectic constituent is preferably hydrophilic to such an extent that it has a solubility in water of at least 5 mg/ml, preferably at least 25 mg/ml, more preferably at least 50 mg/ml. If the composition is well soluble in water, the API can generally be released immediately after administration. In a preferred embodiment, the composition is an immediate release formulation, releasing its contents immediately following administration with >95% of contents being released within 6 hours.

The eutectic constituent is typically depressing the melting point of the mixture when combined with the API salt through hydrogen bonding. In typical embodiments, the eutectic constituent is one or more selected from the group consisting of the following classes: organic acids, phenolic compounds, terpenoids, organic bases, sugars or sweeteners, glycols, amino acids, quaternary ammonium compounds, and derivatives of these classes.

In an embodiment, which may be preferred, the eutectic constituent may include one or more organic acids which may be one of, but not limited to, malic acid, citric acid, lactic acid, fumaric acid, tartaric acid, ascorbic acid, pimelic acid, gluconic acid, acetic acid and/or derivatives thereof such as nicotinamide.

The eutectic constituent may further or alternatively include one or more phenolic compound which may be one of, but not limited to tyramine hydrochloride and vanillin.

Additionally or alternatively, the eutectic constituent may include one or more terpenoid, which may be one of, but not limited to, terpineol and perillyl alcohol.

In another embodiment, the eutectic constituent may include one or more organic base, which may be one of, but not limited to, urea and guanine.

Further, the eutectic constituent may include one or more sugar or sweeteners selected from the group consisting of, but not limited to, sucrose, glucose, fructose, lactose, maltose, xylose, sucrose, inositol, xylitol, saccharin, sucralose, aspartame, acesulfame potassium and ribitol, as well as their phosphates.

Additionally, the eutectic constituent may include one or more amino acids. Suitable amino acids may be selected from, but are not limited to, for example alanine, glutamic acid, glutamate, asparagine, asp artic acid, lysine, arginine, proline and threonine.

In another embodiment, the eutectic constituent may include one or more quaternary ammonium compounds, which may be one of, but not limited to, choline chloride, thiamine mononitrate and carnitine.

The polymer solubilizer generally contributes to the formation and the stability of the eutectic mixture, even before mixing this mixture with the precipitation inhibitor. In addition, the polymer solubilizer facilitates the dissolution of the precipitation inhibitor (PI) in the composition. The polymer solubilizer may comprise one or more plasticizer.

In a typical composition according to the invention, the polymer solubilizer is present in a molar ratio with respect to the hydrophilic eutectic constituent in the range of between 20 to 1 and 1 to 10, preferably in a range of between 15 to 1 and 1 to 2.

Polymer solubilizer are also known as plasticizer and frequently used in the art as pharmaceutical excipients. See for example Sheskey et al. Handbook of Pharmaceutical Excipients 8th Revised edition, Pharmaceutical Press London. A role of the polymer solubilizer in the present invention is to decrease the viscosity of the eutectic mixture and the composition and to present gel formation.

Examples of suitable polymer solubilizers include esters and lactones of organic acids, dicarboxylic acids and esters thereof, esters, ethers and carbonates of diols and triols, and glycols. The one or more esters and/or lactones of organic acids may be selected from diethyl malate, triethyl citrate, tributyl citrate, ethyl lactate, dimethyl succinate, diethyl succinate, glucuronolactone and D-(+)-glucuronic acid γ-lactone. Additionally or alternatively, the polymer solubilizer may include one or more dicarboxylic acids and/or esters of dicarboxylic acids selected from mono-methyl adipate, dimethyl glutarate and mono-methyl glutarate. Additionally or alternatively, the polymer solubilizer may include one or more esters, ethers and carbonates of diols and/or triols selected from glycerol carbonate, propylene carbonate, ethylene carbonate, 1,2-butylene carbonate, glycerol formal, DL-1,2-isopropylidene glycerol, 1-butoxypropan-2-ol, tri(propylene glycol) methyl ether, dipropylene glycol methyl ether acetate, propylene glycol methyl ether acetate, dipropylene glycol methyl ether, 1-methoxy-2-propanol, diethylene glycol monoethyl ether, 3-methoxy-3-methyl-1-butanol, isosorbide dimethyl ether and dianhydro-d-glucitol. Additionally or alternatively, the polymer solubilizer may be a glycol selected from, but is not limited to, propylene glycol, dipropylene glycol, butylene glycol, glycerol, tetraglycol, 1,2-hexanediol, 1,2-butanediol, PEG 400 and polyglycerol.

The precipitation inhibitor in the composition enhances the bioavailability of the API. In other applications, polymeric precipitation inhibitors (PPI), which are specific PIs, have been demonstrated to be useful in improving drug solubility and bioavailability of poorly water-soluble APIs in the gastrointestinal (GI) tract. In this respect reference is made to a publication by Vasconceles et al. in Drug Discovery Today, 2007, Vol 12, pages 1068-1075 and PPIs described there and/or in references therein, acting to reduce API precipitation and thereby creating a supersaturated state resulting in a boost in drug bioavailability of molecules capable of permeating the GI tract. Polymeric PI's are also disclosed in Warren et al., Journal of Drug Targeting, 2010, 18(10), 704-731 and Xu and Dai International Journal of Pharmaceutics 453 (2013) 36-43.

In a preferred embodiment of the present invention, the PI is hydrophilic. The PI is preferably hydrophilic to such an extent that it has a solubility in water of at least 0.1 mg/ml. It is thus possible that the hydrophilicity of the PI is less than that of the eutectic constituent.

Good water-soluble PIs include cyclodextins, which are preferred PIs for use in the present invention. The cyclodextrins useful in the present invention refer to cyclodextrins that are soluble in aqueous medium with pH range below 14. Suitable cyclodextrins may include but are not limited to alpha cyclodextrin, beta cyclodextrin and gamma cyclodextrin. Advantageously, cyclodextrins can provide be used with a duel functionality in a eutectic system, the first function is as a eutectic constituent stabilizing the eutectic mixture and the second is as an excipient that enhances the solubility of the active ingredient.

In addition or alternatively, the PI comprises one or more PPIs. The PPI useful in the present invention refer to polymers that are soluble in aqueous medium at pH range below 14, preferably in the range indicated above for the PI in general. These may be ionic or neutral polymers with polar or charged functional groups. Preferably, the PPI is a water-soluble polymer. Suitable PPI's may be selected from the group consisting of homopolymers and copolymers of N-vinyl lactams, especially homopolymers and copolymers of N-vinyl pyrrolidone, e.g. polyvinyl pyrrolidone (PVP), copolymers of N-vinyl pyrrolidone and vinyl acetate or vinyl propionate, polyvinyl caprolactam-polyvinyl acetate-polyethylene glycol graft copolymers, such as those available under the tradename Soluplus®, block copolymers of ethylene oxide and propylene oxide, also known as polyoxyethylene/polyoxypropylene block copolymers or polyoxyethylene polypropyleneglycol, such as those available under the tradename Poloxamer®, lauroyl polyoxyglycerides cellulose esters and cellulose ethers; in particular methylcellulose, hydroxyalkylcelluloses, in particular hydroxypropylcellulose, hydroxyalkylalkylcelluloses, in particular hydroxypropylmethylcellulose, high molecular polyalkylene oxides such as polyethylene oxide and polypropylene oxide and copolymers of ethylene oxide and propylene oxide, vinyl acetate polymers such as copolymers of vinyl acetate and crotonic acid, partially hydrolyzed polyvinyl acetate (also referred to as partially saponified “polyvinyl alcohol”), polyvinyl alcohol, oligo- and polysaccharides such as carrageenans, galactomannans and xanthan gum, and mixtures of one or more thereof.

In the composition, the PI can be homogeneously mixed within the eutectic mixture and as such, be present in the same phase as the eutectic mixture. In other words, the PI can be dissolved in the eutectic mixture, thereby forming a single liquid phase, which is for instance liquid at 37° C. It was found that as such, precipitation of the API salt can particularly effectively be inhibited. The eutectic mixture can accordingly also be considered a eutectic solvent for the PI.

In a particular embodiment, the composition consists of the eutectic mixture based on the API salt, the eutectic constituent and the polymer solubilizer, and the PI. No additional excipients may be required.

The PI in the pharmaceutical composition in accordance with the present invention is preferably present in a weight ratio with respect to the API salt between 0.2 to 1 and 40 to 1, preferably between 0.35 to 1 and 20 to 1, more preferably between 0.5 to 1 and 10 to 1. This ratio also applies to composition including a plurality of PIs and/or APIs, in which case the ratio relates to all PIs and/or APIs present in the composition. The most suitable ratio depends i.a. on the desirable physiochemical properties of the composition such as viscosity and the desired concentration of the API salt in the composition.

A particularly suitable or optimal PI which suitably or most effectively increases the concentration free drug in solution of the active ingredient when the mixture is exposed to an aqueous environment, can be selected by including the PI in the composition at different concentrations which are similar to that of the API then testing the formulation in dissolution apparatus. The concentration of the API can be analyzed over time and the PI with an effective or even the highest concentration over time can be determined to be preferred. Each PI can be compared with itself at different concentrations e.g. 50 mg/ml and 100 mg/ml and to other PIs at similar concentrations. As used for this purpose, the term ‘dissolution’ refers to test 711 “Dissolution” in United States Pharmacopeia 24, United States Pharmacopeial Convention, Inc., Rockville, Md., 1999 (the “USP”). Various fluids can be used as the dissolution media, including acids, buffers, simulated digestive tract fluids, etc., and many of these are defined in various monographs of the USP. An example of a procedure uses “Apparatus 2,” which has a vessel containing a medium that is stirred with a rotating paddle. Typically, a dosage unit is immersed into the medium and samples of the medium are withdrawn at intervals for drug content analysis, frequently using high performance liquid chromatography (“HPLC”) techniques.

The present invention and its preferred embodiments provides several significant advantages over conventional formulation technologies. Firstly, by including the active ingredient in the formation of the eutectic mixture itself rather than solubilizing it in an excipient a significantly higher concentration can be achieved. This high concentration allows a high payload e.g. 200 mg to be administered in a single capsule (of ˜0.91 ml), this is very beneficial when compared to a low concentration e.g. 10 mg/ml where 20 ml would have to be administered to achieve the same payload. Administering a full dose in a single capsule is highly preferred by industry due to patient compliance, logistic costs and production costs. As such, the pharmaceutical composition of the present invention can suitably be used in a medical treatment comprising enteral administration, i.e. administration is via the human gastrointestinal tract such oral and/or rectal administration of capsules comprising the composition. When comparing to standard solid formulation technologies such as solid dispersions, liquid formulations offer significant benefits such as dose flexibility, enhanced development speed and simpler scale up processes.

To allow enteral administration, the hydrophilic pharmaceutically acceptable eutectic constituent, the polymer solubilizer and the precipitation inhibitor (PI) are each present in the composition in pharmaceutically acceptable amounts. The United States Food and Drug Administration (FDA) has compiled an Inactive Ingredient Database that provides information on inactive ingredients present in FDA-approved drug products.

Furthermore, the bioavailability of the composition through the gastrointestinal (GI) tract allows enteral administration.

As used herein, the singular forms “a”, “an” and “the” are intended to include the plural forms as well, unless the context clearly indicates otherwise. The term “and/or” includes any and all combinations of one or more of the associated listed items. It will be understood that the terms “comprises” and/or “comprising” specify the presence of stated features but do not preclude the presence or addition of one or more other features.

For the purpose of clarity and a concise description features are described herein as part of the same or separate embodiments, however, it will be appreciated that the scope of the invention may include embodiments having combinations of all or some of the features described.

The invention can be illustrated by the following, non-limiting examples.

### EXAMPLE 1

A hydrogen chloride (HCl) salt of itraconazole was prepared and mixed with choline chloride and dipropylene glycol at a molar ratio of 0.25:0.5:2. This mixture was heated to 50° C. and stirred for 30 minutes until a colourless and clear solution formed. This mixture was stable at room temperature over the period of 1 month. There was a significant depression in the melting point of both itraconazole HCL and choline chloride, from 170° C. and 302° C. respectively to less than room temperature (20° C.).

Dow Chemical®—AFFINISOL® HPMC HME 15LV was solubilized in the eutectic mixture at a concentration of 50 mg/ml to enhance the dissolution characteristics of the active ingredient.

### EXAMPLE 2

A hydrogen chloride (HCl) salt of itraconazole was prepared and mixed with xylitol and dipropylene glycol at a molar ratio of 0.25:0.5:2. This mixture was heated to 50° C. for 30 minutes until a colourless and clear solution formed. This mixture was stable at room temperature over the period of 1 month. There was a significant depression in the melting point of both itraconazole HCL and choline chloride, from 170° C. and 302° C. respectively to less than room temperature (20° C.).

Dow Chemical®—AFFINISOL® HPMC HME 15LV was solubilized in the eutectic mixture at a concentration of 50 mg/ml to enhance the dissolution characteristics of the active ingredient.

### EXAMPLE 3

A hydrogen chloride (HCl) salt of 1-adamantanamine hydrochloride was purchased from TCI Chemicals (A0588). The API HCl was mixed with xylitol and dipropylene glycol at a molar ratio of 0.25:0.5:2. This mixture was heated to 50° C. for 60 minutes until a colourless and clear solution formed. This mixture was stable at room temperature over the period of 1 month. There was a significant depression in the melting point of both API HCL and choline chloride, from 280° C. and 302° C. respectively to less than room temperature (20° C.).

Dow Chemical®—AFFINISOL® HPMC HME 15LV was solubilized in the eutectic mixture at a concentration of 50 mg/ml to enhance the dissolution characteristics of the active ingredient.

### EXAMPLE 4

A hydrogen chloride (HCl) salt of 1-Adamantanamine hydrochloride was purchased from TCI Chemicals (A0588). The API HCl was mixed with choline chloride and dipropylene glycol at a molar ratio of 0.25:0.5:2. This mixture was heated to 50° C. for 60 minutes until a colourless and clear solution formed. This mixture was stable at room temperature over the period of 1 month. There was a significant depression in the melting point of both API HCL and choline chloride, from 280° C. and 302° C. respectively to less than room temperature (20° C.).

Dow Chemical®—AFFINISOL® HPMC HME 15LV was solubilized in the eutectic mixture at a concentration of 50 mg/ml to enhance the dissolution characteristics of the active ingredient.

### EXAMPLE 5

A hydrogen chloride (HCl) salt of aminoguanidine hydrochloride was purchased from TCI Chemicals (A0588). The API HCl was mixed with choline chloride and dipropylene glycol at a molar ratio of 0.25:0.5:2. This mixture was heated to 50° C. for 60 minutes until a colourless and clear solution formed. This mixture was stable at room temperature over the period of 1 month. There was a significant depression in the melting point of both API HCL and choline chloride, from 280° C. and 302° C. respectively to less than room temperature (20° C.).

Dow Chemical®—AFFINISOL® HPMC HME 15LV was solubilized in the eutectic mixture at a concentration of 50 mg/ml to enhance the dissolution characteristics of the active ingredient.

### EXAMPLE 6

A hydrogen chloride (HCl) salt of aminoguanidine hydrochloride was purchased from TCI Chemicals (A0588). The API HCl was mixed with xylitol and dipropylene glycol at a molar ratio of 0.25:0.5:2. This mixture was heated to 50° C. for 60 minutes until a colourless and clear solution formed. This mixture was stable at room temperature over the period of 1 month. There was a significant depression in the melting point of both API HCL and choline chloride, from 280° C. and 302° C. respectively to less than room temperature (20° C.).

Sigma Aldrich® now Merck®-Polyvinylpyrrolidone K90, powder, average M_{w} 360.000 g/mol. was solubilized in the eutectic mixture at a concentration of 50 mg/ml to enhance the dissolution characteristics of the active ingredient.

### EXAMPLE 7

A hydrogen chloride (HCl) salt of propanolol hydrochloride was purchased from TCI Chemicals (A0588). The API HCl was mixed with choline chloride and dipropylene glycol at a molar ratio of 0.25:0.5:2. This mixture was heated to 50° C. for 60 minutes until a colourless and clear solution formed. This mixture was stable at room temperature over the period of 1 month. There was a significant depression in the melting point of both API HCL and choline chloride, from 280° C. and 302° C. respectively to less than room temperature (20° C.).

Sigma Aldrich® now Merck®-Polyvinylpyrrolidone K90, powder, average M_{w} 360.000 g/mol. was solubilized in the eutectic mixture at a concentration of 50 mg/ml to enhance the dissolution characteristics of the active ingredient.

### EXAMPLE 8

A hydrogen chloride (HCl) salt of propanolol hydrochloride was purchased from TCI Chemicals (A0588). The API HCl was mixed with choline chloride and dipropylene glycol at a molar ratio of 0.25:0.5:2. This mixture was heated to 50° C. for 60 minutes until a colourless and clear solution formed. This mixture was stable at room temperature over the period of 1 month. There was a significant depression in the melting point of both API HCL and choline chloride, from 280° C. and 302° C. respectively to less than room temperature (20° C.).

Sigma Aldrich® now Merck®-Polyvinylpyrrolidone K90, powder, average M_{w} 360.000 g/mol. was solubilized in the eutectic mixture at a concentration of 50 mg/ml to enhance the dissolution characteristics of the active ingredient.

### EXAMPLE 9

A sodium (Na) salt of Cefazolin was purchased from TCI Chemicals (A0588). The API HCl was mixed with xylitol and tripropylene glycol at a molar ratio of 1:0.5:4. This mixture was heated to 50° C. for 60 minutes until a colourless and clear solution formed. This mixture was stable at room temperature over the period of 1 month. There was a significant depression in the melting point of both Cefazolin Na and Xylitol to less than room temperature (20° C.).

Sigma Aldrich® now Merck®-Polyvinylpyrrolidone K90, powder, average M_{w} 360.000 g/mol. was solubilized in the eutectic mixture at a concentration of 50 mg/ml to enhance the dissolution characteristics of the active ingredient.

### EXAMPLE 10

A disodium (2Na) salt of Fosfomycin was purchased from TCI Chemicals (A0588). The API 2Na was mixed with xylitol and tripropylene glycol at a molar ratio of 1:1:4. This mixture was heated to 50° C. for 60 minutes until a colourless and clear solution formed. This mixture was stable at room temperature over the period of 1 month. There was a significant depression in the melting point of both Fosfomycin 2Na and Xylitol to less than room temperature (20° C.).

Dow Chemical®—AFFINISOL™ HPMC HME 4M was solubilized in the eutectic mixture at a concentration of 50 mg/ml to enhance the dissolution characteristics of the active ingredient.

### EXAMPLE 11

A potassium (K) salt of Lorsartan was purchased from TCI Chemicals (A0588). The API K was mixed with sodium propyl paraben and tripropylene glycol at a molar ratio of 1:0.5:4. This mixture was heated to 50° C. for 60 minutes until a colourless and clear solution formed. This mixture was stable at room temperature over the period of 1 month. There was a significant depression in the melting point of both Lorsartan K and sodium propyl paraben to less than room temperature (20° C.).

Dow Chemical®—AFFINISOL™ HPMC HME 4M was solubilized in the eutectic mixture at a concentration of 50 mg/ml to enhance the dissolution characteristics of the active ingredient.

### EXAMPLE 12

A sodium (Na) salt of Sulfamethazine was purchased from TCI Chemicals (A0588). The API Na was mixed with sodium propyl paraben and tripropylene glycol at a molar ratio of 1:0.7:4. This mixture was heated to 50° C. for 60 minutes until a colourless and clear solution formed. This mixture was stable at room temperature over the period of 1 month. There was a significant depression in the melting point of both Sulfamethazine Na and sodium propyl paraben to less than room temperature (20° C.).

Dow Chemical®—AFFINISOL™ HPMC HME 4M was solubilized in the eutectic mixture at a concentration of 50 mg/ml to enhance the dissolution characteristics of the active ingredient.

### EXAMPLE 13

A hydrogen chloride (HCl) salt of propanolol hydrochloride was produced and mixed with xylitol, sorbitol and triporpylene glycol at a molar ratio of 0.5:0.375:0.375:10. This mixture was heated to 50° C. and stirred for 60 minutes until a colourless and clear solution formed. This mixture was stable at room temperature over the period of 1 month. There was a significant depression in the melting point of both itraconazole HCl, xylitol and sorbitol, from 170° C., 92° C. and 95° C. respectively to less than room temperature (20° C.).

This itraconazole HCl formulation according to the invention was selected to evaluate the in vivo bioavailability of the API in comparison to a commercial itraconazole formulation, Sporanox® (ex Janssen-Cilag SpA; Italy). The goal of this study was to show the impressive performance of the invented technology when dramatically increasing the concentration of the formulation (13× increase) and increasing the dose (2.2× increase) compared to the best available commercial formulation. The data below shows increased bioavailability even when corrected for the administered higher dose.

For this purpose, fasted male Sprague-Dawley rats (n=6) with a mass of ˜300 g, were dosed through oral gavage with 1.5 mg of itrazonacole using the 10 mg/ml Sporanox® oral solution. To compare, an additional cohort of fasted male Sprague-Dawley rats (n=6) were dosed with 3.25 mg of itraconazole HCl through a 130.9 mg/ml F4 formulation dosed in size 9 HPMC capsules according to the present invention.

After dosing, the plasma levels of the API were determined as a function of time. Samples were withdrawn 0, 1, 2, 3, 5, 7, 9, 12, 24 and 48 hours after administering the formulations. API levels were determined by Liquid Chromatography-Mass Spectrometry (LC-MS).

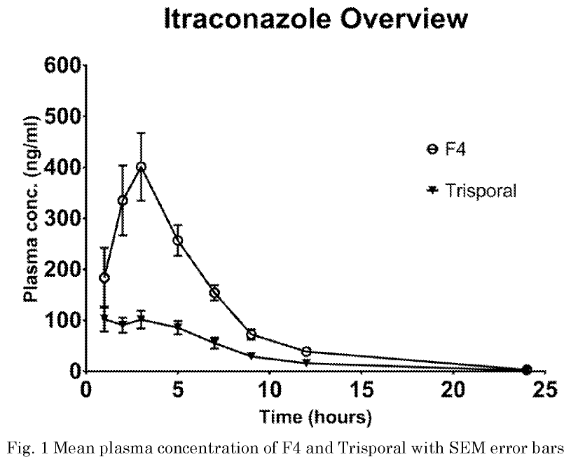

FIG. 1 depicts the mean plasma levels (ng/mL) of itraconazole achieved with Sporanox® compared to formulation F4. F4 achieves a significantly higher concentration in the blood.

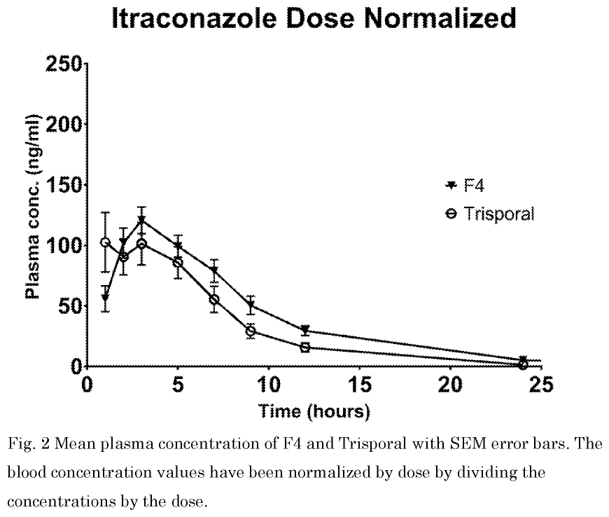

FIG. 2 also depicts the mean plasma levels (ng/ml) of itraconazole achieved with Sporanox® compared to formulation F4. In this graph, the bioavailability has been normalized to dose. Importantly this graph shows that the bioavailability of itraconazole is increased when increasing the concentration of the formulation 13-fold. This is particularly important as bioavailability typically reduces significantly as the concentration of the formulation increases.

1. A pharmaceutical composition that is liquid at 37° C. and 1 atm, said composition comprising providing a eutectic mixture of at least an active pharmaceutical ingredient (API) salt, a hydrophilic pharmaceutically acceptable eutectic constituent and a polymer solubilizer, and further comprising a precipitation inhibitor (PI), wherein said hydrophilic pharmaceutically acceptable eutectic constituent, polymer solubilizer and precipitation inhibitor are each present in the composition in pharmaceutically acceptable amounts.

2. The pharmaceutical composition according to claim 1, wherein the API salt is based on a basic API and an acid, which acid comprises an atomic anion, and wherein said hydrophilic pharmaceutically acceptable eutectic constituent comprises one or more functional groups capable of hydrogen bonding or, wherein the API salt is based on a basic API and an acid, which acid comprises a molecular anion, and wherein said hydrophilic pharmaceutically acceptable eutectic constituent comprises a small moiety capable of forming strong intermolecular bond.

3. The pharmaceutical composition according to claim 1, wherein the API salt is based on an acidic API and a base, which base comprises an atomic cation and wherein said hydrophilic pharmaceutically acceptable eutectic constituent comprises one or more functional groups capable of hydrogen bonding.

4. The pharmaceutical composition according to claim 1, wherein the pharmaceutically acceptable eutectic constituent comprises one or more organic acids, phenolic compounds, terpenoids, organic bases, sugars or sweeteners, glycols, amino acids, quaternary ammonium compounds, or derivatives of these classes or combinations thereof.

5. The pharmaceutical composition according to claim 1, wherein said polymer solubilizer comprises one or more esters and/or lactones of organic acids, one or more dicarboxylic acids and/or esters of dicarboxylic acids, one or more esters, ethers and carbonates of diols and/or triols, one or more glycols or combinations thereof.

6. The pharmaceutical composition according to claim 1, wherein said PI is a polymeric precipitation inhibitor (PPI) that comprises a polymer that is soluble in an aqueous medium at a concentration of greater than 0.1 mg/ml at a pH value in the range of 1 to 8.

7. The pharmaceutical composition according to claim 1, wherein said PI comprises one or more cyclodextrins that are soluble in an aqueous medium at a concentration of greater than 0.1 mg/ml at a pH value in the range of 1 to 8.

8. The pharmaceutical composition that is preferably liquid at claim 1, wherein the precipitation inhibitor is solubilized and homogeneously mixed within the eutectic mixture.

9. (canceled)

10. The pharmaceutical composition according to claim 1, wherein the API is present in a concentration of at least 50 mg/ml based on the total composition.

11. The pharmaceutical composition according to claim 1, wherein the weight ratio of PI to API is between 0.2 to 1 and 40 to 1.

12. The pharmaceutical composition according to claim 1, wherein the molar ratio of the polymer solubilizer to the eutectic constituent is in the range of 20 to 1 and 1 to 10.

13. The pharmaceutical composition according to claim 1 for use in a medical treatment comprising enteral administration.

14. (canceled)

15. A method for preparing a pharmaceutical composition according to claim 1 comprising providing a eutectic mixture of at least an active pharmaceutical ingredient (API) salt, a hydrophilic pharmaceutically acceptable eutectic constituent and a pharmaceutically acceptable amount of a polymer solubilizer, and mixing said eutectic mixture with a pharmaceutically acceptable amount of a precipitation inhibitor (PI) to form said pharmaceutical composition.

16. The pharmaceutical composition according to claim 1 wherein said polymer solubilizer comprises one or more esters and/or lactones of organic acids selected the group consisting of diethyl malate, triethyl citrate, tributyl citrate, ethyl lactate, dimethyl succinate, diethyl succinate, glucuronolactone and D-(+)-glucuronic acid γ-lactone; one or more dicarboxylic acids and/or esters of dicarboxylic acids selected from the group consisting of mono-methyl adipate, dimethyl glutarate and mono-methyl glutarate; one or more esters, ethers and carbonates of diols and/or triols selected from the group consisting of glycerol carbonate, propylene carbonate, ethylene carbonate, 1,2-butylene carbonate, glycerol formal, DL-1,2-isopropylideneglycerol, 1-butoxypropan-2-ol, tri(propylene glycol) methyl ether, dipropylene glycol methyl ether acetate, propylene glycol methyl ether acetate, dipropylene glycol methyl ether, 1-methoxy-2-propanol, diethylene glycol monoethyl ether, 3-methoxy-3-methyl-1-butanol, isosorbide dimethyl ether and dianhydro-d-glucitol; or one or more glycols selected from the group consisting of propylene glycol, dipropylene glycol, butylene glycol, glycerol, tetraglycol, 1,2-hexanediol, 1,2-butanediol, PEG 400 and polyglycerol; or combinations thereof.

17. The pharmaceutical composition according to claim 1 wherein said PI is a polymeric precipitation inhibitor (PPI) that comprises a polymer selected from the group consisting of homopolymers and copolymers of N-vinyl lactams, especially homopolymers and copolymers of N-vinyl pyrrolidone, e.g. polyvinyl pyrrolidone (PVP), copolymers of N-vinyl pyrrolidone and vinyl acetate or vinyl propionate, polyvinyl caprolactam-polyvinyl acetate-polyethylene glycol graft copolymers, such as Soluplus®, block copolymers of ethylene oxide and propylene oxide, also known as polyoxyethylene/polyoxypropylene block copolymers or polyoxyethylene polypropyleneglycol, such as Poloxamer®, lauroyl polyoxyglycerides cellulose esters and cellulose ethers; in particular methylcellulose, hydroxyalkylcelluloses, in particular hydroxypropylcellulose, hydroxyalkylalkylcelluloses, in particular hydroxypropylmethylcellulose, high molecular polyalkylene oxides such as polyethylene oxide and polypropylene oxide and copolymers of ethylene oxide and propylene oxide, vinyl acetate polymers such as copolymers of vinyl acetate and crotonic acid, partially hydrolyzed polyvinyl acetate (also referred to as partially saponified “polyvinyl alcohol”), polyvinyl alcohol, oligo- and polysaccharides such as carrageenans, galactomannans and xanthan gum, and mixtures of one or more thereof.

18. The pharmaceutical composition according to claim 1 wherein said PI comprises one or more cyclodextrins selected from the group consisting of alpha cyclodextrin, beta cyclodextrin and gamma cyclodextrin.

19. The pharmaceutical composition according to claim 1 wherein the API is present in a concentration of at least 100 mg/ml, based on the total composition.

20. The pharmaceutical composition according to claim 1 wherein the weight ratio of PI to API is between 0.35 to 1 and 20 to 1.

21. The pharmaceutical composition according to claim 1 wherein the API is present in a concentration of at least 150 mg/ml based on the total composition, and wherein the weight ratio of PI to API is between 0.5 to 1 and 10 to 1.
